(19) United States
(12) Patent Application Publication
(10) Pub. No.: US 2011/0111399 A1
(43) Pub. Date: May 12, 2011

(54) Methods And Compositions Including Diagnostic Kits For The Detection of Staphylococcus Aureus

(76) Inventors: Shawn Mark O'Hara, Richboro, PA (US); Mark James Kopnitsky, Lenhartsville, PA (US)

(21) Appl. No.: 12/809,086

(22) PCT Filed: Dec. 23, 2008
(86) PCT No.: PCT/US08/88121

§ 371 Date: Jan. 13, 2011

Related U.S. Application Data
(60) Provisional application No. 61/008,776, filed on Dec. 24, 2007.

(51) Int. Cl. C12Q 1/68

(52) U.S. Cl. 435/6

(57) ABSTRACT

Methods and compositions, including diagnostic kits, for the detection of Staphylococcus Aureus (SA) and clinically important antibiotic resistant forms thereof, such as methicillin-resistant Staphylococcus aureus (MRSA), vancomycin-resistant Staphylococcus aureus (VRSA), mupirocin-resistant Staphylococcus aureus (mupSA), and the like, from individuals in a sample population are disclosed Also disclosed are cost effective methods and kits for bacterial sampling and analysis via inherent and expeditious SA cell disruption methods followed by Direct PCR, circumventing the need, expense and contamination πsks associated with DNA isolation methods These improved methods in conjunction with SA prevalence analysis are applied so as to eliminate the approximately 70% of samples in the human population which do not carry SA (SA negative), followed by a second more costly test for antibiotic resistant forms thereof, such as amplification to confirm for presence of MRSA or other target disease

### CROSS REFERENCE TO RELATED APPLICATIONS

This application is a non-provisional application, which is incorporated by reference herein and claims priority, in part, of U.S. Provisional Application No. 61/008,776, filed 24 Dec. 2007.

### BACKGROUND OF THE INVENTION

1. Field

The present invention relates to novel methods and compositions, including diagnostic kits, for the detection of Staphylococcus Aureus (SA) and antibiotic resistant forms thereof, such as known clinically important forms including methicillin-resistant Staphylococcus aureus (MRSA), vancomycin-resistant Staphylococcus aureus (VRSA), mupirocin-resistant Staphylococcus aureus (mupSA), variants of the foregoing and the like, from individuals in a sample population.

2. Background Art

Staphylococcus Aureus (SA) is a major cause of skin, soft tissue, and bloodstream infections in patients, causing conditions that may rapidly become fatal if not treated effectively. SA and methicillin-resistant Staphylococcus aureus (MRSA) are now endemic in many hospitals in the United States and other countries. In the United States, the incidence of disease from antibiotic resistant forms of SA is expected to continue to increase. Recently, the Centers for Disease Control and Prevention (CDC) demonstrated that by 2005 there were more deaths related to invasive MRSA disease than from HIV-AIDS. According to the CDC about 30 percent of the general population carries SA, of which about 3 percent carries MRSA, and in health care settings such as hospitals, the percentage of SA which is MRSA may vary from 3-60%.

Colonization (defined as carriage only from topological origin such as nasal, nasopharyngeal, inguinal, anal, ear or other topological site combination), not blood infection with SA, MRSA, VRSA etc. Colonization is associated with eventual infection. These infections have high medical care cost and poor clinical outcome. With an increased burden of in-hospital MRSA-related disease and the emerging concern that community-associated (CA)-MRSA continues to increase, medical professionals and the public are urgently seeking a rapid and cost effective means to limit the spread of these pathogens. In addition, a number of state legislatures have passed, and more are considering, legislation to require active surveillance for MRSA. The CDC study indicated that 85% of invasive MRSA infections are still healthcare-associated, suggesting that hospital programs can be effective in stopping this epidemic.

SA has become the single leading pathogen in health care-associated infections. Nasal carriage of SA has been postulated as a source of bacteremia, surgical-site, and other infections and a reservoir of SA in hospitals. Early detection of nasal carriage (colonization) and cost effective diagnosis has been shown to prevent the spread of infections, reduce transmission and reduce net hospital costs.

Screening patients for SA colonization using culture methods is time consuming and generally requires 1 to 4, or even more, days for accurate detection and identification of SA. However, it is possible to obtain results within two (2) hours using real-time polymerase chain reaction (PCR) assays in detecting SA (see, for example, “Direct Detection of Staphylococcus aureus from Adult and Neonate Nasal Swab Specimens Using Real-Time Polymerase Chain Reaction,” Paule, S. M., Pasquariello A. C., Hacek, D. M. Fisher A. G., Thomson, R. B., Kaul, K. L., and Peterson, L. R., J. Molecular Diagnostics, Vol. 6, No. 3, pgs. 191-196, 2004) and “New Real-Time PCR Assay for Rapid Detection of Methicillin-Resistant Staphylococcus aureus Directly from Specimens Containing a Mixture of Staphylococci,” A. Huletsky, R. Giroux, V. Rossbach, M. Gagnon, M. Vaillancourt, M. Bernier, F. Gagnon, K. Truchon, M. Bastien, F. J. Picard, A. van Belkum, M. Ouellette, P. H. Roy, and M. G. Bergeron, J. Clin. Micro, Vol. 42, No. 5, pgs, 1875-1884, May 2004).

Consequently, a cost-effective method for the rapid detection of SA would provide a needed diagnostic tool in the detection, prevention, and treatment of this contagious disease. PCR assays to detect nasal colonization of SA have the potential to obtain information in less than 1 hour. A rapid PCR assay as a first step in a population sampling strategy to screen patients for SA would enable significant cost savings, especially when screening for the antibiotic resistant forms of SA such as MRSA, VRSA and the like.

It is known that Methicillin resistance in S. aureus is caused by the acquisition of an exogenous gene, mecA, that encodes an additional B-lactam-resistant penicillin-binding protein (PBP), termed PBP 2a (or PBP2′). The mecA gene is carried by a mobile genetic element, designated staphylococcal cassette chromosome rnec (SCCmec), inserted near the chromosomal origin of replication. The SCCmec DNAs are integrated at a specific site (attBscc) in the methicillin-susceptible S. aureus (MSSA) chromosome.

Applications for detecting MRSA using nasal swabs and real-time PCR testing have increased the speed and accuracy for identification of SA and confirmation of its antibiotic resistant forms such as MRSA, VRSA and the like. Multiplex PCR, incorporating the detection of the mecA and fernA genes, has been used in diagnosis of MRSA from colonies isolated from nasal cultures. Similarly, this multiplex approach has been used successfully for identifying MRSA directly from mixed staphylococcus nasal swab samples following immunomagnetic enrichment of SA from these nasal samples (see, for example. “Rapid Detection of Methicillin-Resistant Staphylococcus aureus Directly from Sterile or Nonsterile Clinical Samples by a New Molecular Assay,” Patrice Francois, Didier Pittet, Manuela Bento, Be'atrice Pepey, Pierre Vaudaux, Daniel Lew, and Jacques Schrenzel, J. Clin Micro, Jan. 2003, Vol. 41, No. 1, pgs. 254-260).

The burden of SA infections on hospitals in the United States has recently been demonstrated in reports showing that SA infections were reported in patient discharge diagnosis for 0.8% of all hospital inpatients, or 292,045 stays per year. In-patients with SA infection had, on average, 3 times the length of hospital stay than inpatients without this infection (14.3 vs 4.5 days; P=0.001), 3 times the total charges ($48 vs $14; P=0.001), and 5 times the risk of in-hospital death (11.2% vs 2.3%; Pa0.001). Even when controlling for hospital-fixed effects and for patient differences in diagnosis-related groups, age, sex, race, and co-morbidities, the differences in mean length of stay, total charges, and mortality were significantly higher for hospitalizations associated with SA. The potential benefits to hospitals in terms of reduced use of resources and costs as well as improved outcomes from preventing SA, MRSA and VRSA infections are significant. Several hospitals have successfully implemented control strategies, some by using PCR, however the exorbitant costs of those tests are impeding their broader utilization. The high costs of the current FDA approved tests are primarily due to sample preparation and special equipment designed to eliminate carryover and crossover contamination (See Table 1, infra), and because 70% of the samples could be ruled-out by using a much less expensive test.

More recently, PCR procedures for identifying the SA SCCrnec insertion site have enabled the detection of MRSA directly from mixed Staphylococcal nasal samples without the need for SA enrichment or colony isolation. It is also important to note that the SCCrnec approach has an inherent 5% false positive rate. Also recently the US FDA approved two versions of the SCCmec PCR assay, as shown in Table 1, infra. However, broad adoption and active surveillance by healthcare providers using these conventional SCCinec-based assays has not been accomplished, due primarily because these assays are viewed as too costly. The high overall cost of MRSA screening using these conventional SCCmec assays is primarily due to their relatively elaborate sample preparation methods and their lack of test population stratification, as 70-75% of samples can be ruled out with a much less expensive and rapid test for SA-positive sample stratification prior to a subsequent rapid MRSA verification test. Thus, in spite of the availability of accurate MRSA PCR assays, there still exists a need to provide cost-effective and rapid detection of SA for subsequent use in diagnostic assays for the antibiotic resistant forms thereof.

### SUMMARY OF THE INVENTION

The present invention provides novel methods and compositions, including diagnostic kits, which, when compared with largely conventional techniques, are capable of providing cost-effective management and control tools for the detection and diagnosis of SA and its known antibiotic resistant forms, and variants thereof.

The present invention therefore relates to novel methods and compositions, including diagnostic kits, for the detection of Staphylococcus Aureus (SA) and antibiotic resistant forms thereof, such as those which are known to be clinically important, including methicillin-resistant Staphylococcus aureus (MRSA), vancomycin-resistant Staphylococcus aureus (VRSA), mupirocin-resistant Staphylococcus aureus (mupSA), variants of the foregoing and the like, from individuals in a sample population.

The present invention provides more cost effective methods and kits for bacterial sampling and analysis via inherent and expeditious SA cell disruption methods followed by Direct PCR, circumventing the need, expensive and contamination risks associated with DNA isolation methods. Direct PCR of the sample using cell disruption without DNA isolation provides a faster and less expensive screening method for SA in laboratory and point-of-care settings than conventional procedures. These improved methods of the invention in conjunction with SA prevalence analysis are applied so as to eliminate the approximately 70% of samples in the human population which do not carry SA (SA negative), followed by a second more costly test for antibiotic resistant forms thereof, such as amplification to confirm for presence of MRSA or other target disease.

Accordingly, it is an objective of the present invention to provide methods and compositions for improved sample preparation methods and diagnostic kits compared with those of the conventional art, for enabling the direct transfer of SA disrupted cells from nasal swab samples directly into SA-PCR reactions, thereby employing DNA amplification without the laborious costly steps of SA DNA isolation, known in the art as “direct PCR”.

It is a further objective of the of the present invention to provide diagnostic kits including improved nasal swab sampling methods for staphylococcus DNA preparation, thereby providing more accurate amplification results with conventional techniques such as PCR.

It is another objective of the present invention to provide an improved and preferably a more cost effective population-based stratification algorithm, employing SA-PCR to first eliminate samples which do not carry SA (SA negative=70-75%), followed by screening the remaining SA positive samples (25-30%) for antibiotic resistance, such as MRSA and the like.

It is yet another objective of the present invention to provide improved methods and compositions for accomplishing the foregoing based upon population prevalence of SA and its antibiotic resistant forms

It is a still further objective of the present invention to provide methods and compositions which incorporate the foregoing stated objectives in a repetitive, reliable and efficient manner, to make use of direct PCR of the sample, and to provide faster and less expensive screening methodologies for SA in laboratory and point-of-care settings, with minimal cost.

### THE DRAWINGS

FIG. 1A is an illustration of results from the procedures described in Example 1 herein, in accordance with the present invention.

FIG. 1B is also an illustration of results from the procedures described in Example 1 herein, in accordance with the present invention.

FIG. 1C is a flow chart depicting population screening with SA PCR detection in accordance with the present invention, using a DNA derived from a mucosal sample without isolation of the sample DNA from disrupted SA cells, followed by antibiotic resistant testing only for the remaining 25-30% of SA positive samples.

FIG. 2 shows graphical representations of results achieved in the performance of methods in accordance with the present invention as described in Example 2 herein.

FIG. 3 shows graphical representations of results from PCR analysis obtained in accordance with the invention as described in Example 4.

### DETAILED DESCRIPTION OF THE PREFERRED EMBODIMENTS

The present invention has been developed to streamline sample preparation and utilize SA prevalence, in order to provide more cost effective diagnostic methods, compositions, and diagnostic kits. Table 1, below, shows a comparison of commercially available MRSA assays:

TABLE 1
Comparison of Commercial MRSA Assays

| | | BD |
|---|---|---|
| Feature | Cepheid GeneXpert $45 ea. | GeneOhm* $30 ea |
| Instrumentation | Cepheid GeneXpert | PCR |
| | Dx System | equipment |
| Fluidics | Self-contained and | Multiple |
| | automated after swab elution | manual steps |
| | and 2 single-dose reagents | |
| Lysis | Sonication (automated | Glass beads |
| | single-use cartridge) | (manual) |
| DNA Target | Sequences incorporating the | Sequence |
| | Sequence insertion site | near the insertion |
| | (AttBssc) of SCCmec | site of SCCmec |
| Internal Controls | Sample processing control | Internal |
| | and probe check control | control |
| Time to Result | 75 minutes | 60 to 75 |
| | | minutes |
| Users | Operators with little clinical | CLIA high |
| | lab experience to experienced | complexity |
| | lab technologists | technologists |

*Originally marketed as the IDI-MRSA.
Source: FDA 510(k) summary

In contrast to the above conventional assays, the methods and compositions of the present invention utilize the sampling algorithm and Direct PCR from SA disrupted nasal swabs as samples in a FDA approved PCR kit. It is believed that direct nasal SA DNA sample preparation without DNA isolation for PCR is capable of providing a potentially faster and less expensive screening method than the afore-described conventional techniques, for SA in health care settings. A further preferred embodiment of the present invention focuses on population prevalence of SA relative to MRSA, VRSA, ORSA, or CONS/CoNS. For example, SA has been determined to be well established and prevalent in the general population, at around 30% compared to MRSA which is approximately 0.8%. In hospitals, SA prevalence remains at approximately 30% while the proportion of MRSA can increase dramatically within its SA population, potentially rising to 60% of the SA population. The present invention provides an improved strategy for MRSA screening utilizing direct PCR for the much simpler and cheaper SA analysis, resulting in a 3 to 4 times less expensive test than current, commercially available FDA approved MRSA PCR kits. The less expensive SA PCR test is used to rule-out 70% of the samples, which is SA negative, resulting in an overall 50% MRSA screening savings. These savings can be passed on to the consumer to enable a much more cost effective screening paradigm. With lower costs, broader implementation becomes possible, resulting in a significant reduction in healthcare system costs due to MRSA, as well as a reduction in morbidity.

In accordance with the present invention, determination of SA negative samples is assessed by conventional direct PCR. Direct PCR in the general sample set is accomplished by an initial bacterial cell wall disruption. Surprisingly, it has been discovered that SA cell disruption and thus amplifiable DNA often exists naturally in nasal mucus samples, which can be readily captured via nasal swabs. Equally surprising, it has been further discovered in accordance with the invention that heating or freezing the nasal swab mucus sample, either by itself or in aqueous based buffers, will further increase the proportion of disrupted SA cells and thus amplifiable DNA. Furthermore, these cells which are disrupted naturally by the nasal mucosal defense mechanisms and or by freeze thaw and heating have been found to be capable of providing amplifiable SA DNA at diagnostically relevant levels compared to the gold standard of culture detection. SA cell disruption can further be accomplished through enzymatic cell wall lysis, achromopeptidase preparations (ACP—a mixture of at least 4 proteinases) proteinase K, Lysozyme, autolysin, sonication wave energy (sonication), electrolysis, pulsed electric field (PEF), electroporation, bead mill homogenizers, centrifugation, ionic or non-ionic detergents, combinations of any of the foregoing, or by any means of successful SA cell disruption known in the art. Accordingly, it is to be appreciated that the present invention contemplates and includes all such techniques, including but not limited to inherent natural lysis, high temperature lysis, low temperature lysis, electroporation, sonication, bead mill, Saponin, quaternary alkyl amines such as NIMBUS, nisin antibiotic, and combinations thereof.

Further, in accordance with the invention elimination of PCR inhibitors can be accomplished by utilization of agents such as IgG(s), mucin(s), glycoproteins, nasal RX, blood, heat denaturation, activated charcoal, activated carbon, rapid hybridization, or by any means known in the art. The present invention also preferably contemplates use of a nasal sample SA immunomagnetic procedure prior to cell wall disruption followed by direct PCR, which can include such techniques known to those skilled in the art such as immunomagnetic enrichment with protein A antibodies, IgG bead binding to SA protein A, thermostable nuclease nuc antibodies, coagulase antibodies, fibronectin FN binding, fibronectin surface binding protein(s), or combinations thereof.

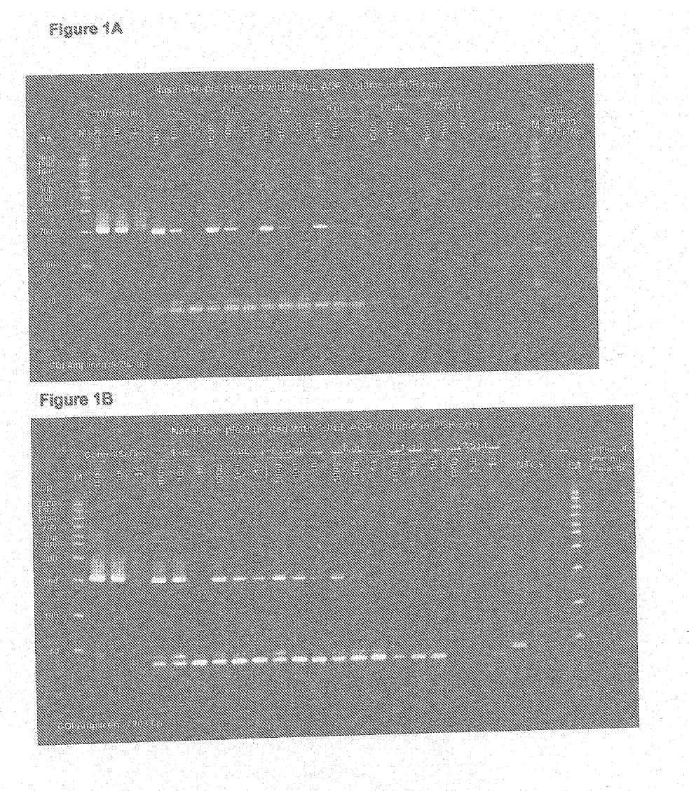

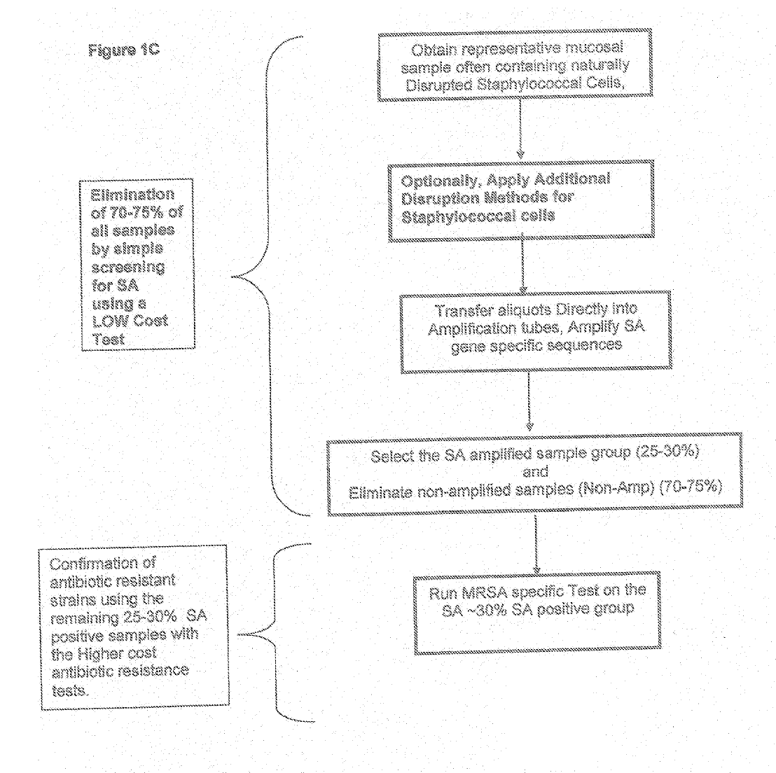

DNA extraction and isolation, accomplished by means known to those skilled in the art, can be combined in accordance with the present invention with the selection algorithm such as set forth in FIG. 1C, and also is considered, instead of a direct PCR, as useful in a preferred embodiment of the present invention.

It is to be also appreciated that the genes targeted in any of the amplification steps of the practice of the present invention include those well known in the art for SA or MRSA identification, for example, femA, nuc, sa442, or tufA can be used as SA specific genes. For example, for SA, immunomagnetic detection of mupirocin resistance uses ileS-2. Coagulase negative Staphylococcus (CONS) are endogenous to humans topologically and all mucus membranes such as nasal mucosa and can be considered as an inherent target for an overall process control in these SA methods and kits, especially applying the tufA specific gene targets.

Amplification assays contemplated for use in the present invention include, but are not limited to, DNA amplification assays, PCR assays incorporating thermostable polymerases, and isothermal amplifications methods. It is to be appreciated that one skilled in the art may conceive of various suitable amplification methods that will be useful in the practice of the present invention, and that therefore the invention is not intended to be limited thereby.

As mentioned previously, SA direct PCR, when provided in the practice of the present invention, enables a more cost effective and rapid screening test compared to conventional tests, such as the currently FDA-approved MRSA PCR tests, initially, it has been found that SA direct PCR will identify SA carriers to rule-out approximately 70% of the general sample population pool (MRSA′VRSA suspect population), resulting in approximately a 50% reduction in screening costs. This improved screening algorithm, outlined in FIG. 1C, results in significant cost savings and as such provides broader screening and with fewer SA/MSSA/MRSA/VRSA associated deaths. Thus, the present invention provides cost saving improvements over current PCR antibiotic resistant SA screening tests, especially for MRSA and VRSA. These improvements involve, in part, the incorporation of “direct” nasal SA sample preparation methods applied in combination with a selection process for MRSA and VRSA. This selection process utilizes bacterial population demographics such as, but not limited to, the data suggesting that only about 30% of the human population at any one time has nasal colonization with SA. Direct nasal SA sample preparation involves the disruption and liberation of bacterial genomic DNA, specifically SA genomic DNA, but without DNA extraction. Instead of purifying DNA, a disrupted sample is directly transferred to a SA specific PCR reaction mix for testing. The direct sample prep results in a significant savings in total testing time before a result is obtained, reduction in operator hands-on time and a reduction in the reagents/equipment normally used to extract/isolate genomic DNA. The significant reduction in operator hands-on time not only achieves significant measurable cost savings and time to results, it also significantly reduces overall assay complexity and thus contamination potential due to less open tube manipulations.

Although the present invention has been described in some detail, the following examples are also provided by way of illustration and for purposes of clarity of understanding, and it will be readily apparent to those of ordinary skill in the art in light of the teachings of this invention as set forth herein that certain changes and modifications may be made thereto without departing from the spirit or scope of the invention.

### Example 1

### Achromopepticiase Disruption of the SA Cell Wail is Compatible with Direct-PCR & Nasal Swab Samples Contain PCR Inhibitors

Nasal samples were obtained from nasal swabs after elution with 200 micro liters of TE. Samples were then incubated with or without achromopeptidase (ACP) incubation at 1 Unit/ul at 37 degrees C. for 15 minutes followed by 99 degrees C. for 5 minutes. Direct TaqMan PCR amplification of an exogenous spiked in control template DNA at a volume of up to 2.5 micro liters of this ACP lysate in a 25 micro liter PCR reaction confirmed compatibility. Further, transfer of volumes greater than 2.5 ul in to the 25 ul PCR showed inhibition from both sample types suggesting that inhibition might start to negatively affect PCR above this volume proportion if not removed. The results are illustrated in FIG. 1A and FIG. 1B. Thus in accordance with this procedure of the present invention, ACP Direct PCR from nasal swab samples can be improved by removal of PCR inhibitors using methods such as cell or DNA enrichment, adsorption to activated charcoal etc.

### Example 2

### QIAamp DNA Isolation Using ACP Lysis Substituted in Qiagen's Protocol for the Proteinase K ALT Lysis

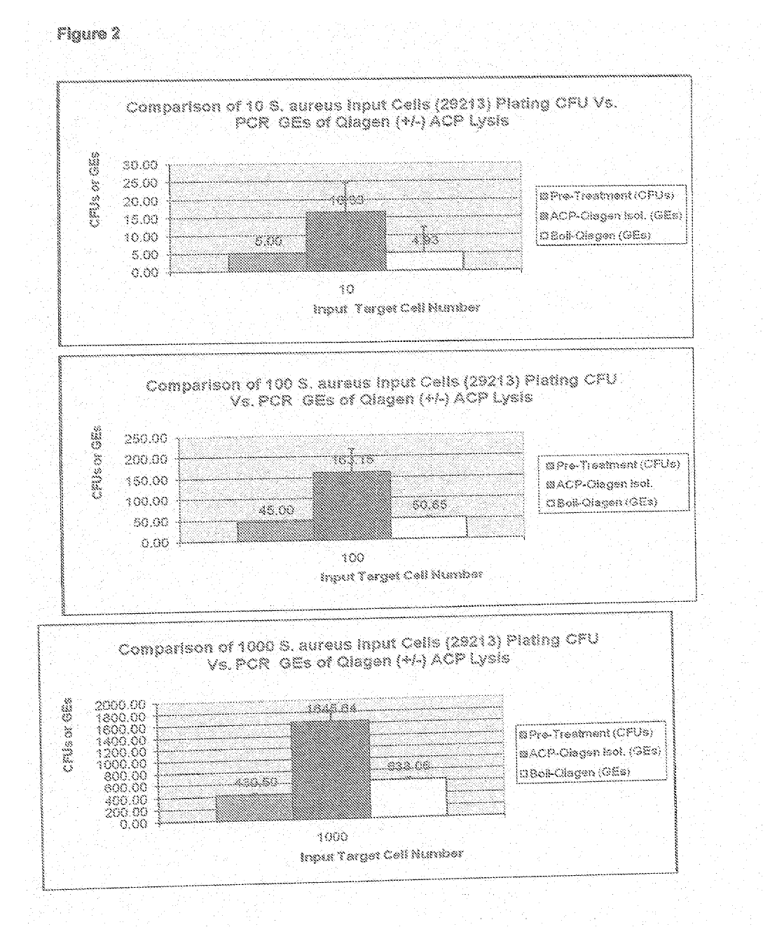

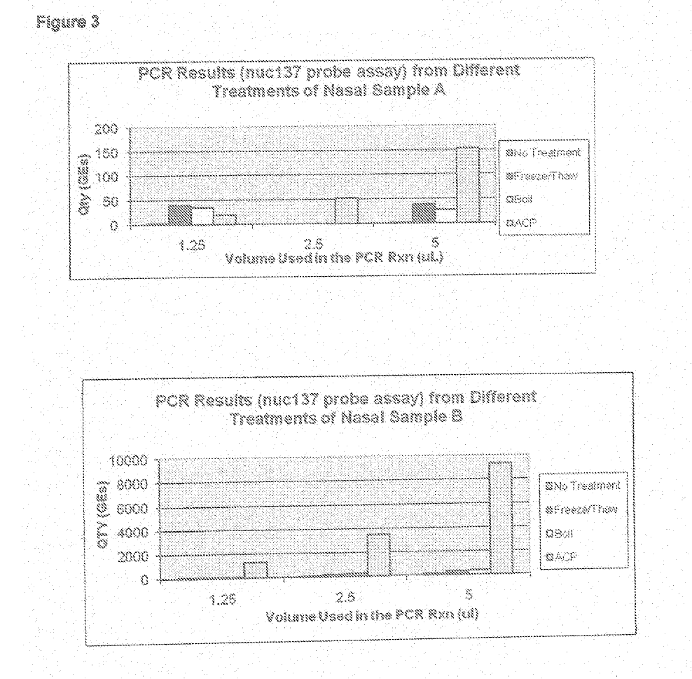

ACP SA cell lysis was used in conjunction with the commercially available Qiagen Silica DNA Isolation QiAamp kit, available from Qiagen, Inc., by substituting ACP cell wall lysis steps performed in accordance with the present invention in place of the Qiagen protocol specified Proteinase K lysis steps. In brief, the ACP disruption system described in Example 1 was performed in duplicate in TE buffer spiked with varying bacterial colony plate forming unit numbers (CFUs) using SA strain ATCC-29213. The ACP lysed bacteria was then input into the y QIAamp DNA Micro kit isolation protocol found the handbook published by Qiagen and dated August 2003 on page 35, starting at step 5. As shown in FIG. 2, the graph targeting 10 input cells shows a reproducible SA lower limit of genomic DNA copy number equivalents (GEs) measured by TaqMan nuc137 real-time quantitative PCR of less than or equal to 10 CFU. The ACP treated sample was split prior to the Qiagen isolation procedure—one portion of the split was plated resulting in no CFU demonstrating that all SA were dead due to cell wall lysis (data not shown). The ACP lysed samples in accordance with the instant procedure consistently scored higher than boil-Qiagen (ACP Lysis without the ACP enzyme added) showing that boiling for 5 minutes lyses SA but not nearly as efficiently as when combined with ACP enzyme preparation, and also out scored CFU, which is consistent with the known clustering culture behavior of SA which was visible under a microscope from 1-10 cells CFU. Likewise ACP treated SA titrations at these same levels followed by Direct-gPCR GEs values were found to outperform parallel CFU measurements. These sample amplification results are consistent with, and suggest that, the vast majority of SA cell walls are also disrupted by this ACP treatment in accordance with the invention, liberating PCR amplifiable genomic DNA.

### Example 3

### Prevalence of Nasal SA by Culture & PCR

In a preliminary study, using routine SA culture methods (commercially available Becton Diekinson(BD)-CHROMagar-SA & latex agglutination) in parallel with quantitative PCR scoring 2 independent SA-specific gene targets (femA-SA) previously published primers (2003 Francois et al.), and thermostable nuclease gene (nuc) assay specificity were verified. Swab samples were taken from 15 randomly selected subjects, from the anterior nares of the subjects using an Ames single headed rayon swab. One swab from each nare were designated left nare=L and right nare=R. Each swab sample was directly streaked on tryptic soy agar blood plate (TSA BAP) and on CHROMagar-SA, commercially available from BD. After direct streaking each swab was then eluted in 200 ul TE (10 mM, 1 mM EDTA) by vortexing for 1 minute prior to ACP lysis and DNA isolation using the Qiagen Micro kit identical to that used in Example 2. After ACP lysis followed by Qiagen isolation, TaqMan qPCR was performed. The culture was called positive only if suspect colonies were biochemically confirmed using a BD BBL Staphyloslide latex agglutination test for S. aureus. PCR was called positive only if a Ct value was less than 40 cycles as determined relative to a linear external standard curve. Process blanks and controls indicated no contamination present during this study. Data from the foregoing is shown on Table 2 below. TaqMan PCR results showed 4/15 (27%) samples positive for presence of SA and all four were positive for both femA-SA and nuc-137 PCR assays. Both types of culture plates were also in agreement and were confirmed by latex agglutination test for proteinA/coagulase. Thus all 4 tests were concordant and the SA nasal carriage prevalence was 27% in agreement with the literature values ranging from about 20-30%.

Subsequently, TadMan nuc qPCR was performed on 30 independent subjects. For this study, the sample preparation was modified to eliminate the DNA isolation component leaving only nasal swab elute, ACP lysis followed directly by qPCR. Results, shown in Table 2 below, showed 5/30 (17%) positive SA prevalence level.

TABLE 2

| SA | |
|---|---|
| CHROMagar | PCR Quantity in GEs |

| Sample | CFU | Nuc-137 | femA-SA |
|---|---|---|---|
| 1L | Negative | 0 | 0 |
| 1R | Negative | 0 | 0 |
| 2L | Negative | 0 | 0 |
| 2R | Negative | 0 | 0 |
| 3L | 4 Positive | 21 | 21 |
| 3R | Negative | 0 | 1 |
| 4L | Negative | 0 | 0 |
| 4R | Negative | 0 | 0 |
| 5L | Negative | 0 | 0 |
| 5R | Negative | 0 | 0 |
| 6L | 1 Positive | 0 | 0 |
| 6R | 7 Positive | 5 | 6 |
| 7L | 37 Positive | 3 | 3 |
| 7R | 26 Positive | 1947 | 2435 |
| 8L | Negative | 0 | 0 |
| 8R | Negative | 0 | 0 |
| 9L | Negative | 0 | 0 |
| 9R | Negative | 0 | 0 |
| 10L | Negative | 0 | 0 |
| 10R | Negative | 0 | 0 |
| 11L | 115 Positive | 3545 | 3443 |
| 11R | 13 Positive | 1820 | 1635 |
| 12L | Negative | 0 | 0 |
| 12R | Negative | 0 | 0 |
| 13L | Negative | 0 | 0 |
| 13R | Negative | 0 | 0 |
| 14L | Negative | 0 | 0 |
| 14R | Negative | 0 | 0 |
| 15L | Negative | 0 | 0 |
| 15R | Negative | 0 | 0 |

### Example 4

### Disrupted Nasal Swab Derived SA by Boiling, Freeze Thawing, Inherent to Nasal Mucosal Flora

Further disruption methods through boiling, freeze thawing and the possibility of an inherently amplifiable SA DNA were evaluated from nasal swab derived SA specimens in combination with Direct PCR. With the persistently positive and negative nasal SA carriage subjects identified in Example 3, the above-established ACP disruption method was compared to 3 new disruption sample preparation methods for compatibility with Direct-PCR. Each of 4 subjects (2 positive & 2 negative) was swabbed and then eluted by vortexing into TE yielding 300 ul of TE swab eluate. 50 ul of eluate was then disrupted for each the following 4 methods: ACP, boiling, freeze thawing and no treatment (or inherent to sample). 1.25 ul of each of these 4 treatments was then transferred to a 25 ul SA specific nuc137 TaqMan real-time PCR reaction and amplified for 45 cycles relative to standard curve. A no template & master mix control & process blanks were run for the entire process. All contamination controls were found to be negative for nuc137. The 2 previous SA negative samples were found to be again negative for all 4 treatments via nuc137 (data not shown). The 2 previous SA positive were found to be both positive by Direct-PCR for ALL 4 treatments including the untreated “inherent” samples FIG. 3. As further illustrated in FIG. 3, this demonstrates that PCR amplifiable DNA are inherent to nasal mucosal SA and likely all flora, and that in accordance with the present invention ACP yielded a significant improvement and commercially enabling method of sample preparation for Direct-PCR.

### Example 5

### Immunomagnetic Enrichment

Immunomagnetic enrichment prior to sample disruption and Direct PCR is also contemplated for use in the practice of the present invention and may be expected to improve Direct PCR by eliminating potential PCR inhibitors. Thus any protocol that enriches for the SA bacteria live or dead or the nucleic acids thereof will in theory improve the analytical sensitivity and accuracy of the Direct PCR approach.

### Example 6

### Consequences of Identifying Persistently Positive/Negative Groups

The majority of SA carriage positive and negative individuals are persistently so, at a constant of approximately a 30% prevalence rate. It is believed that this persistent prevalence rate is due to some as yet uncharacterized human factor(s). Thus, once these persistent positive and negative groups are identified, the need to actively test the general population may be reduced to about the 30% persistent rate plus a minor group of transitory individuals.

While certain of the preferred embodiments of the present invention have been described and specifically exemplified above, it is not intended that the invention be limited to such embodiments. Various modifications may be made thereto without departing from the spirit and scope of the present invention, and the full scope of the improvements provided by the invention are delineated in the following claims.

What is claimed is:

1. In a method for the detection of Staphylococcus Aureus (SA) and antibiotic resistant forms thereof, the improvement comprising:
a. obtaining a sample from a subject suspected of containing SA cells;
b. disrupting SA cells present in said sample;
c. directly transferring said SA disrupted cells from said sample directly into means for performing SA-PCR reactions; and
d. analyzing the results of said SA-PCR reactions.

2. The method of claim 1 wherein the antibiotic resistant form of SA is selected from the group consisting of MRSA, VRSA, mupSA or variants thereof.

3. The method of claim 1 wherein said sample is a nasal swab sample, nasopharyngeal swab, inguinal, anal, ear or any topological sample.

4. The method of claim 1 wherein said analyzing comprises a population-based stratification algorithm.

5. The method of claim 1 wherein said analyzing further comprises screening for SA positive samples for antibiotic resistance.

6. A kit for the detection of Staphylococcus Aureus (SA) and antibiotic resistant forms thereof, comprising:
a. a sample;
b. means for disrupting SA cells present in said sample;
c. means for directly transferring said SA disrupted cells from said sample directly into means for performing SA-PCR reactions; and
d. means for analyzing the results of said SA-PCR reactions.

7. The kit of claim 6, further comprising means for screening SA positive samples for antibiotic resistance.
